(19) United States
(12) Patent Application Publication
(10) Pub. No.: US 2004/0013627 A1
(43) Pub. Date: Jan. 22, 2004

(54) Propylene glycol hyaluronate esters and composition comprising the same

(75) Inventors: Hideki Hirai, Tokyo (JP); Michiki Asai, Tokyo (JP)

Correspondence Address: BROWDY AND NEIMARK, P.L.L.C., 624 NINTH STREET, NW, SUITE 300, WASHINGTON, DC, 20001-5303, US

(73) Assignee: KIBUN FOOD CHEMIFA CO., LTD, Tokyo (JP)

(21) Appl. No.: 10/406,589

(22) Filed: Apr. 4, 2003

Related U.S. Application Data
(63) Continuation-in-part of application No. 09/607,929, filed on Jun. 30, 2000.

(30) Foreign Application Priority Data
Dec. 14, 1999 (JP) 355160/1999

(51) Int. Cl. A61K 7/06; A61K 7/11; A61K 7/075; A61K 7/08; C08B 37/00

(52) U.S. Cl. 424/70.13; 424/70.21; 536/53

(57) ABSTRACT

Disclosed are propylene glycol hyaluronate esters having a limiting viscosity of 14-35 dL/g and an esterification degree of 10-90% as well as skin conditioning methods, moisturizing methods and emulsifying methods using said propylene glycol hyaluronate esters. Propylene glycol hyaluronate esters of the present invention are compounds showing excellent viscosity stability in low-pH systems and cation-containing systems and also showing high emulsifiability, hydration power, moisturizing effect and liposome stabilizing effect.

### FIELD OF THE INVENTION

The present invention relates to propylene glycol hyaluronate esters and compositions comprising the esters, such as an agent for external use to skin, which refers to “skin preparations for external use” hereinafter. The composition of the present invention can be widely used as cosmetic and pharmaceutical products because of their excellent viscosity stability, emulsifiability, hydration power, moisturizing effect and loposome stabilizing effects.

### RELATED ART

Dry skin is caused by excessive loss of moisture from the surface of the skin exposed to dry air or cleansing. In these days, various environmental chemical substances also inhibit skin functions such as lipid secretion to often invite dry skin. Therefore, there is a demand for a skin preparation for external use to prevent dry skin and provide an excellent moisturizing effect.

Various active compounds having a moisturizing effect have been provided, which are mainly based on water-soluble polyols. Some of them including propylene glycols have already been commercialized. However, many of commercialized moisturizing compounds are associated with uncomfortable feel during application or insufficient moisturizing effect, so that there is still a demand for the development of a new moisturizing compound.

An alternative moisturizing compound is sodium hyaluronate, which draws special interest as a valuable compound because of high hydration effect. However, aqueous solutions of sodium hyaluronate have a disadvantage in stability, which is high at neutral pH range but lowered in acidic solutions or salt solutions. This leads to the problem that the moisturizing effect cannot be effectively produced under some storage conditions or application conditions during use as cosmetics or the like, and there is a need for a solution thereto.

In view of these problems of the prior art, our studies were devoted to provide a material that stably exists even in low-pH solutions or salt solutions and has excellent emulsifiability and hydration effect. Thus, an object of the present invention is to provide a compound that can be widely used as cosmetic and pharmaceutical products because of excellent viscosity stability, emulsifiability, hydration power and moisturizing effect.

In the field of cosmetic and pharmaceutical products, often employed is a method of applying pharmaceutical ingredients to skin in the form of skin preparations for external use thereof for percutaneous absorption of pharmaceutical ingredients. However, even when pharmaceutical ingredients are applied to skin in the form of such skin preparations for external use, they could not be satisfactorily percutaneously absorbed in many cases. This is because the outermost layer of skin, which is referred to as a skin keratin layer, has a physiological function as a barrier that prevents invasion of external impurities into bodies. A skin preparation for external use which has been prepared merely by incorporating a pharmaceutical ingredient into a base material could not sufficiently penetrate through such a skin keratin layer.

To solve the problem, adding various subcutaneous absorption promoters to the base material for skin preparations for external use is a general technique carried out these days. For examples of subcutaneous absorption promoters, known are dimethylsulfoxide, dimethylformamide, dimethylacetamide, methyldecyl sulfoxide, etc. However, the effect of these subcutaneous absorption promoters is unsatisfactory and, in addition, they are unfavorable from the viewpoint of safety as they too much irritate skin. Other various subcutaneous absorption promoters have heretofore been proposed. For example, JP-A 61-27966 discloses a subcutaneous absorption promoter that comprises a 1-substituted azacycloalkan-2-one such as 1-n-hexylazacyclopentan-2-one or 1-n-heptylazacyclo-pentan-2-one; JP-A 63-208536 and 63-208537 disclose a subcutaneous absorption promoter that comprises at least two different types of specific surfactants combined; JP-A 9-157129 discloses a method of using guanidine derivatives or their acid addition salts, α-monoglyceryl ethers, urea, etc.; JP-A 2001-288233 discloses a method of using block copolymers having a backbone chain of polyethylene glycol; and JP-A 2001-278981 discloses a method of using block copolymers having a polysiloxane moiety and a hydrophobic functional group. However, most subcutaneous absorption promoters that have heretofore been proposed do not always satisfy the two requirements of good subcutaneous absorption-promoting effect and safety to skin. In general, for example, some of them that have a good subcutaneous absorption-promoting effect are not always safe to skin, while some others that are safe to skin do not always have a good subcutaneous absorption-promoting effect.

On the other hand, it is known that liposome has a subcutaneous absorption-promoting effect. Liposome is a closed vesicle formed of two molecules of a natural lipid such as phospholipid or glycolipid or a similar synthetic lipid, in which the hydrophobic terminals of the two molecules overlap with each other with the hydrophilic terminals thereof facing outside to form a membrane structure. The aqueous phase in side the vesicle or inside the bimolecular membrane may envelop an active ingredient such as a pharmaceutical ingredient therein. In addition, since the bimolecular membrane structure is similar to the structure of cell membrane, it has a high affinity for body membranes and therefore exhibits an excellent subcutaneous absorption. Further, since liposome is a closed vesicle, the pharmaceutical ingredient enveloped therein is hardly influenced by the external environment. Therefore, even unstable pharmaceutical ingredients that could not be incorporated into ordinary preparations could be stably incorporated into liposomes for administration. In addition, if it becomes possible to control the degradation of liposomes, it will be possible to control the release of pharmaceutical ingredient enveloped in liposomes, and if so, liposomes may be applied to drug deliver system (DDS). JP-A 52-151718 says that the stability of hemoglobin enveloped in liposomes is improved; and JP-A 52-143218 says that when the surfaces of liposomes are modified, then the effect of the pharmaceutical ingredients enveloped therein toward the target cells can be controlled. Further, JP-A 51-86117 discloses a slow-release preparation that comprise liposomes.

The lipid to constitute liposomes is essentially phospholipid. Phospholipid is an essential ingredient to constitute body membranes and is indispensable to bodies. We daily take phospholipid from foods, and phospholipid is used in various fields as an additive to foods, cosmetics, medicines, etc. The safety of phospholipid is extremely high. Accordingly, so far as natural lipid such as phospholipid is used for them, it may be said that liposomes are not so much toxic or immunogenic to bodies but are highly safe thereto. However, the chemical and physical stability of liposomes formed of phospholipid is poor, and this is one reason for which the practical use of liposomes is difficult. Specifically, since liposomes are formed of lipid molecules bonding to each other via weak hydrophilic bonds, their structures are readily broken by physical factors such as temperature, pressure, pH change, stirring, etc. Still other problems with liposomes are that, since they are unstable to various physiologically-active substances, they could not be incorporated into cosmetics that comprise many different compounds, and the conditions for enveloping active ingredients into them are limited, and, in addition, since their long-term stability is poor, they may cause discoloration or offensive smells of products. Moreover, in living bodies, liposomes are readily degraded by the action of a lipid-degrading enzyme such as phospholipase, and in blood, the structure of liposomes is readily broken as phospholipid is released from them by the action of lipoprotein. Further, liposomes are englobed by the phagocytosis of immunocytes such as macrophages, and are degraded by the action of the liposome-degrading enzyme in the cells. Because of these reasons, at present, the application range of liposomes is extremely limited.

For increasing the stability of liposomes, some methods have heretofore been provided. In particular, it is known that, when the surfaces of liposomes are coated with a certain type of additive, then the stability of the thus-coated liposome increases. One example of the method comprises adding any of trehalose, maltose, sucrose, glucose, lactose or dextran to liposomes to stabilize them, as in JP-T 62-500102 and 62-501631 (the term “JP-T” as used herein means a published Japanese translation of a PCT application). For increasing the stability of liposomes, JP-A 7-108166 discloses a method of stabilizing liposomes by adding a trehalose fatty acid ester thereto; and JP-A 58-201711 and 58-49311 disclose a method of coating liposomes with an ester of a polysaccharide of any of amylopectin, pullulan, dextran, dextran sulfate, chitosan or pullulan sulfate and a fatty acid of any of lauric acid, myristic acid, palmitic acid or stearic acid. These methods may improve the stability of liposomes but are extremely disadvantageous for industrial application since they require a special technique for preparing the derivatives. Accordingly, it is still necessary to find out some substances that are effective for improving the stability of liposomes.

On the other hand, also known is a method of coating liposomes with a hyaluronic acid derivative. For example, JP-A 3-143540 and JP-B 7-55961 disclose a method of coating liposomes with an acylated hyaluronic acid. For the method, however, an acyl group or an amido group is introduced into the alcoholic hydroxyl group of hyaluronic acid, and the reaction to give the derivative takes a long time and is therefore still disadvantageous for industrial application.

An object of the present invention is to provide a simple means for stabilizing liposomes that envelop various effective ingredients therein. Another object of the invention is to stabilize liposomes and to improve the absorbability and the effect duration of the active ingredients enveloped in the liposomes in bodies. Still another object of the invention is to provide a composition, especially a skin preparation for external use that contains the stabilized liposomes.

### SUMMARY OF THE INVENTION

As a result of careful studies to attain the above object, we accomplished the present invention on the basis of the finding that propylene glycol hyaluronate esters satisfying specific conditions have excellent properties.

Accordingly, the present invention provides propylene glycol hyaluronate esters having a limiting viscosity of 14-35 dL/g and an esterification degree of 10-90%. The limiting viscosity is preferably 14-33.5 dL/g. The esterification degree is preferably 10-65%, more preferably 15-65%, still more preferably 15-40%, even more preferably 20-40%.

Propylene glycol hyaluronate esters of the present invention are preferably combined with imidazoline-based amphoteric surfactants such as 2-lauryl-N-carboxymethyl-N-hydroxyethyl imidazolinium betaine and 2-alkyl-N-carboxy-methyl-N-hydroxyethyl imidazolinium betaine.

The present invention also provides skin preparations for external use containing said propylene glycol hyaluronate esters. The skin preparations for external use may comprise loposome. Skin preparations for external use according to the present invention are useful as moisturizers and emulsifiers, especially as emulsifiers for low-pH systems, emulsifiers for cation-containing systems and high-hydration emulsifiers. Skin preparations for external use according to the present invention are also useful as a loposome stabilizer.

The present invention also provides a method for stabilizing liposome which comprises adding to a composition comprising liposome a propylene glycol hyaluronate ester having a limiting viscosity of 14-35 dL/g and an esterification degree of 10-90%. The present invention also provides a skin conditioning method which comprises applying a composition containing the propylene glycol hyaluronate ester to the skin; a skin moisturizing method which comprises applying a composition containing the propylene glycol hyaluronate ester to the skin; and a rough-skin treatment method which comprises applying a composition containing the propylene glycol hyaluronate ester to the skin.

### BRIEF DESCRIPTION OF THE DRAWING

FIG. 1 shows microscopic photographs of the emulsion compositions prepared in Test Example 15.

### DETAILED DESCRIPTION OF THE INVENTION

Propylene glycol hyaluronate esters and skin preparations for external use according to the present invention will now be explained in detail. It should now be noted that, in this specification, any notation using a word “to” or “-” indicates a range defined by values placed before and after such word, where both ends of such range are included as minimum and maximum values.

Propylene glycol hyaluronate esters of the present invention have a limiting viscosity of 14-35 dL/g and an esterification degree of 10-90%. The limiting viscosity is preferably 14-33.5 dL/g. The esterification degree is preferably 10-65%, more preferably 15-65%, still more preferably 15-40%, even more preferably 20-40%.

As used herein, the “esterification degree” means the proportion of esterified carboxylates among those forming hyaluronic acid. The limiting viscosity of propylene glycol hyaluronate esters can be determined according to the method of Laurentetal. (T. C. Laurentetal. Biochem. Biophys. Acta, 42(1960)476-485).

The type and structure of the hyaluronic acid moiety forming propylene glycol hyaluronate esters of the present invention are not specifically limited. The molecular weight of hyaluronic acid, which is a polysaccharide having repeating units of a disaccharide consisting of D-glucuronic acid and N-acetyl-D-glucosamine, is not specifically limited so far as the limiting viscosity of the resulting propylene glycol esters falls within said range. Hyaluronic acid used herein may be synthesized or purified from natural origins by known means. Substituents on D-glucuronic acid and N-acetyl-D-glucosamine forming hyaluronic acid may be partially derivatized unless the effects of the present invention are excessively hindered. For example, hydroxyl groups may be substituted by alkoxy or other groups. These substitutions can be appropriately carried out within the scope of those skilled in the art.

In propylene glycol hyaluronate esters of the present invention, acid moieties and ester moieties may be localized in their molecules or may be widely distributed. However, those having two or more molecules of hyaluronic acid crosslinked via propylene glycol are excluded.

Processes for preparing propylene glycol hyaluronate esters of the present invention are not specifically limited. An especially preferred process involves reacting a mixture of hyaluronic acid and sodium hyaluronate with propylene oxide. More specifically, sodium hyaluronate is first partially converted into hyaluronic acid in the presence of a hydrochloric acid/ethanol solution or the like and then the reaction mixture is washed with ethanol to give a mixture of hyaluronic acid and sodium hyaluronate. Then, this mixture is esterified with a solution of propylene oxide in ethanol. Preferably, the temperature of the esterification reaction here is 50-80° C. and the reaction time is about 1-10 hours. After reaction, the reaction product may be washed with ethanol, neutralized with a solution of sodium acetate in ethanol, washed with ethanol again, and then dried. According to this process, a propylene glycol hyaluronate ester having the intended effects of the present invention can be efficiently prepared.

Propylene glycol hyaluronate esters of the present invention are quite useful as ingredients of skin preparations for external use. Propylene glycol hyaluronate esters of the present invention are compounds showing excellent viscosity stability in low-pH systems and cation-containing systems and also showing high emulsifiability, hydration power and moisturizing effect. Thus, skin preparations for external use containing a propylene glycol hyaluronate ester of the present invention are useful as moisturizers, emulsifiers for low-pH systems, emulsifiers for cation-containing systems and high-hydration emulsifiers.

Skin preparations for external use according to the present invention were found to have especially high stability and excellent moisturizing effect so that they provide appropriate moisture to the surface of the skin to keep smoothness. That is, skin preparations for external use according to the present invention can keep moisture in the skin for a long period. Such an effect of the present invention is especially remarkable in propylene glycol hyaluronate esters satisfying the conditions described above. It could not be expected that such compounds are much effective than similar hyaluronate esters departing from the conditions described above.

The invention provides a liposome stabilizer that contains a propylene glycol hyaluronate ester having a limiting viscosity of 14-35 dL/g and an esterification degree of 10-90%. The invention also provides a composition (especially a skin preparation for external use) that contains a propylene glycol hyaluronate ester having a limiting viscosity of 14-35 dL/g and an esterification degree of 10-90% and liposomes. The invention further provides a method for stabilizing liposomes, which comprises adding a propylene glycol hyaluronate ester having a limiting viscosity of 14-35dL/g and an esterification degree of 10-90% to a liquid containing liposomes.

Not specifically defined, the lipid that constitutes the liposomes for use in the invention may be any and every one capable of forming a bimolecular lipid membrane structure. For example, it includes natural phospholipids and their derivatives such as egg yolk lecithin, soybean lecithin, hydrogenated egg yolk lecithin, hydrogenated soybean lecithin; acyl-modified synthetic phospholipids such as dimyristoylphosphatidylcholine, dipalmitoylphosphatidylcholine, distearoylphosphatidylcholine, dioleylphosphatidylcholine, dimyristoylphosphatidylethanolamine, dipalmitoylphosphatidylethanolamine, dimyristoylphosphatidic acid, dipalmitoylphosphatidic acid, distearylphosphatidic acid, dioleylphosphatidic acid, dimyristoylphosphatidylglycerol, dipalmitoylphosphatidylglycerol, distearylphosphatidylglycerol; as well as phosphatidylinositol, phosphatidylserine, etc. Any of these are employable herein with no specific limitation.

For preparing the liposomes for use in the invention, employable is any ordinary method of producing liposomes. For example, many methods are known such as voltexing, sonication, pre-vesiculation, ethanol injection, French press, ether injection, annealing, W/O/W emulsification, reversed-phase evaporation, etc. Any of these methods are employable herein, and the invention is not limited to these.

In preparing them, the liposomes for use in the invention may optionally contain a membrane stabilizer. For this, for example, any of sterols such as cholesterol, and, as a charging substance, a fatty acid and a fatty acid salt such as palmitic acid and sodium palmitate may be added to them.

When the propylene glycol hyaluronate ester of the invention is applied thereto, it is believed that liposomes are coated with the ester and are thereby stabilized. Though not wedded to any theory, the liposomes-stabilizing effect of the propylene glycol hyaluronate ester of the invention will be because of the following reasons: The propylene glycol hyaluronate ester is so designed that the carboxyl groups of hyaluronic acid are partly substituted with propylene glycol. Accordingly, hyaluronic acid is highly hydrable, but its propylene glycol ester is made hydrophobic in some degree. Owing to this characteristic thereof, it is believed that the somewhat hydrophobic part of the propylene glycol ester will hydrophobically interact with liposomes while, on the other hand, the major hydrophilic part of the ester will take the water molecules around the liposomes into it as a hydrated gel, and, as a result, each liposome is enveloped in the ester and its stability is thereby improved. Such an excellent liposomes-stabilizing effect is peculiar to the propylene glycol hyaluronate ester that satisfies the condition of the invention, and it could not be foreseen from any conventional knowledge.

The pharmaceutical ingredients that may be enveloped in the propylene glycol hyaluronate ester-coated liposomes of the invention are, for example, vegetable extracts such as hamamelis, peony, matricaria, chamomile; amino acids such as glycine, histidine, serine, and their derivatives; proteins such as hemoglobin, albumin, globulin; anti-inflammatory agents such as oligopeptides, peptides, glycyrrhizin and its salts, glycyrrhetinic acid and its salts, allantoin, epsilon-aminocaproic acid and its salts; vitamins such as α-carotene, β-carotene, ascorbic acid, tocopherol; antioxidants such as tannic acid, flavonoids; nucleotides such as NADH; metal chelating agents such as EDTA, etc. One or more of these may be used herein either singly or as combined. Needless-to-say, any other substances than these are also employable herein.

The propylene glycol hyaluronate esters for use in the invention are highly safe, like sodium hyaluronate, and therefore they give liposomes of high safety not detracting from the high biocompatibility of liposomes. Accordingly, the liposomes thus stabilized with such propylene glycol hyaluronate ester are favorable for ordinary cosmetics and medicines such as skin preparations for external use.

The stabilized liposomes that are produced according to the invention have many applications for pharmaceutical ingredient carriers, and owing to their high subcutaneous absorption-promoting effect, the liposomes enable effective administration of the enveloped pharmaceutical ingredient to bodies. Accordingly, the stabilized liposomes of the invention enable to produce excellent cosmetic and pharmaceutical preparations, and, in addition, they are expected to exhibit an additional effect owing to the synergistic effect of the propylene glycol hyaluronate esters and the enveloped substances, as will be demonstrated in the Examples mentioned below.

Concretely, the stabilized liposomes of the invention are expected to have many applications, for example, for antiaging, skin activation, acne prevention, anti-inflammation, analgesic, lipocatabolism, circulatory system improvement, hair growth promotion, keratin softening, antioxidation, moisturizing, skin whitening, dry and rough skin moisturizing and smoothing, etc. The stabilized liposomes of the invention may be used for any of these applications, but are not limited to them. Accordingly, the propylene glycol hyaluronate ester-coated liposomes of the invention are widely usable in various fields of medicines, medicated preparations for external use, cosmetics, etc.

For example, skin preparations for external use according to the present invention can be used as cosmetic or pharmaceutical products, such as toilet soaps, shampoos, face washes, rinses, eye creams, eye shadows, creams and/or emulsions, lotions, perfumes, face powders, cosmetic oils, cosmetic products for hair and scalp, hair dyes, solid perfumes, powders, packs, shaving creams, shaving lotions, suntan oils, sunscreen oils, suntan lotions, sunscreen lotions, suntan creams, sunscreen creams, foundations, powder perfumes, cheek colors, mascaras, eyebrow colors, nail creams, nail enamels, nail enamel removers, hair washes, bath cosmetics, lip colors, lip creams, eyeliners, dentrifrices, deodorant products, eaux de cologne, hair growers, etc. Skin preparations for external use according to the present invention may also be used as ointments or fomentations.

Skin preparations for external use according to the present invention may also contain various ingredients other than said propylene glycol hyaluronate esters depending on the purpose of use such as improvement of emollient effect, improvement of feel of use, moderation of dehydration after use, improvement of solubility, improvement of emulsifiability, improvement of emulsification stability, improvement of compatibility with oily ingredients, moderation of feel of stretch after use, improvement of skin fit, improvement of spreadability on the skin, moderation of greasiness, prevention of dry skin, enhancement of skin-improving effect, improvement of skin-protecting effect, keratin improvement, normalization of epidermal keratinization (prevention of parakeratosis, prevention of acanthosis and inhibition of disorder of epidermal lipid metabolism via promoted turnover of the skin), moderation of xeroderma such as senile xeroderma, improvement of dry skin conditions such as crack or desquamation, inhibition of the formation of wrinkles, removal of wrinkles, wound healing, prevention and improvement of pigmentation, antiaging, moderation of dandruff or itch, moderation of loss of hair, prevention and treatment of scalp diseases, improvement of setting, improvement of softness, improvement of elasticity, glossing, suppression of melanogenesis, prevention of sunburn, etc.

Depending on the purpose of use, skin preparations for external use according to the present invention may appropriately contain other ingredients such as fats and oils, phospholipids, UV absorbers, IR absorbers, emulsifiers, surfactants, preservatives, antifungal agents, antioxidants, whitening agents, vitamins, amino acids, hormones, peptides, bioactive plant extracts, fluorescent materials, pigments, dyes, perfumes, scrubbing agents, sequestrants, binders, fillers, thickeners, sugars, nutrient ingredients, pH modulators, chelating agents, antibacterials, keratin improvers, keratolytic agents, antibiotics, skin penetration enhancers, blood circulation promoters, antiphlogistics, cytotonic agents, antiinflammatory agents, analgesics, skin softeners, emollients, wound healing agents, metabolism enhancers, etc. Additional moisturizing ingredients other than propylene glycol hyaluronate esters of the present invention may also be contained.

Suitable fats and oils for use in skin preparations for external use according to the present invention include fatty acids such as oleic acid, behenic acid, isostearic acid, lauric acid, myristicacid, palmitic acid, stearic acid, behenic acid, linolic acid, γ-linolenic acid, columbic acid, eicosa-(n-6,9,13)-trienoic acid, arachidonic acid, α-linolenic acid, tymnodonic acid, hexaenoic acid; ester oils such as penta-erythritol-tetra-2-ethyl hexanoate, isopropyl myristate, butyl stearate, hexyl laurate, octyldodecyl myristate, diisopropyl adipate, diisopropyl sebacate; waxes such as beeswax, spermaceti, lanolin, carnauba wax, candelilla wax, vaseline; animal and plant oils such as mink oil, olive oil, castor oil, cacao butter, palmoil, cod liver oil, beef tallow, butter fat, evening primrose oil, rice bran oil, squalane; mineral oils such as hydrocarbon oils, liquid paraffin; silicone oils such as methyl phenyl silicone, dimethyl silicone; higher alcohols such as lauryl alcohol, stearyl alcohol, oleyl alcohol, cetyl alcohol, 2-octyl dodecanol, 2-decyl tetradecanol and derivatives thereof. Suitable organic acids include α-hydroxy acid, hydroxycarboxylic acid, dicarboxylic acid, glycyrrhizic acid, glycyrrhetic acid, mevalonic acid (mevalolactone).

Suitable phospholipids for use in skin preparations for external use according to the present invention include monoacylester-type glycerophospholipids and diacylester-type glycerophospholipids. Specific examples include lysophosphatidylcholine, lysophosphatidylethanolamine, lysophosphatidylserine, lysophosphatidylinositol, phosphatidylcholine, phosphatidylethanolamine, phosphatidylinositol, phosphatidylserine, phosphatidylglycerol, phosphatidic acid, sphingomyelin. Naturally derived lecithins such as yolk and hydrogenates of the compounds mentioned above may also be used.

Suitable UV absorbers for use in skin preparations for external use according to the present invention include oxybenzone (2-hydroxy-4-methoxybenzophenone), oxybenzonesulfonic acid, oxybenzonesulfonic acid (trihydrate), guaiazulene, ethylene glycol salicylate, octyl salicylate, dipropylene glycol salicylate, phenyl salicylate, homomenthyl salicylate, methyl salicylate, methyl diisopropylcinnamate, cinoxate (2-ethoxyethyl p-methoxycinnamate), glyceryl mono-2-ethylhexyl-di-p-methoxycinnamate, 2,2′-di-hydroxy-4-methoxybenzophenone, sodium 2,2′-dihydroxy-4-methoxybenzophenone-5,5′-disulfonate, 2,4-dihydroxybenzophenone, 2,3,4,4′-tetrahydroxybenzophenone, p-aminobenzoic acid, ethyl p-aminobenzoate, glyceryl p-aminobenzoate, amyl p-dimethylaminobenzoate, 2-ethylhexyl p-dimethylaminobenzoate, p-hydroxyanisol, 2-ethylhexyl p-methoxycinnamate, isopropyl p-methoxycinnamate, diisopropyl cinnamate ester, 2-(2-hydroxy-5-methylphenyl)benzotriazole, sodium 2-hydroxy-4-methoxybenzophenone-5-sulfonate, 4-tert-butyl-4′-methoxybenzoylmethane, 2-ethylhexyl salicylate, glyceryl p-monobenzoate, methyl orthoaminobenzoate, 2-hydroxy-4-methoxybenzophenone, amyl p-dimethylaminobenzoate, 2-phenylbenzimidazol-5-sulfonic acid, 2-hydroxy-4-methoxybenzophenone-5-sulfonic acid, dicaproyl trioleate, 2-ethoxyethyl p-methoxycinnamate, butylmethoxy-dibenzoyl-methane, glyceryl mono-2-ethylhexanoyl-di-p-methoxybenzophenone, 2-ethylhexyl-2-cyano-3,3′-diphenylacrylate, 2,2′-dihydroxy-4-methoxybenzophenone, ethyl 4-bishydroxypropyl aminobenzoate.

Suitable emulsifiers and surfactants for use in skin preparations for external use according to the present include nonionic surfactants, anionic surfactants, cationic surfactants and amphoteric surfactants.

Examples of nonionic surfactants include sorbitan esters such as sorbitan monolaurate, sorbitan monooleate, sorbitan monoisostearate; polyoxyethylene sorbitan esters such as polyoxyethylene sorbitan monoisostearate, polyoxyethylene sorbitan monolaurate, polyoxyethylene sorbitan monooleate; glycerol ethers such as glycerol monoisostearate, glycerol monomyristate; polyoxyethylene glycerol ethers such as polyoxyethylene glycerol monoisostearate, polyoxyethylene glycerol monomyristate; polyglycerin fatty acid esters such as diglyceryl monostearate, decaglyceryl decaisostearate, diglyceryl diisostearate; glycerin fatty acid esters such as glyceryl monocaprate, glyceryl monolaurate, glyceryl monomyristate, glyceryl monopalminate, glyceryl monooleate, glyceryl monostearate, glyceryl monolinoleate, glyceryl monoisostearate, glyceryl monodilinoleate, glyceryl monodicaprate; polyoxyethylene glycerin fatty acid esters such as polyoxyethylene glyceryl monomyristate, polyoxyethylene glyceryl monooleate, polyoxyethylene glyceryl monostearate; polyoxyethylene branched alkyl ethers such as polyoxyethylene octyldodecyl alcohol, polyoxyethylene-2-decyltetradecyl alcohol; polyoxyethylene alkyl ethers such as polyoxyethylene oleyl alcohol ether, polyoxyethylene cetyl alcohol ether; polyoxyethylene hydrogenated castor oil fatty acid esters such as polyoxyethylene hydrogenated castor oil, polyoxyethylene dihydrocholesterol ether, polyoxyethylene hydrogenated castor oil isostearate; polyoxyethylene alkyl aryl ethers such as polyoxyethylene octyl phenol ether.

Examples of anionic surfactants include salts of higher fatty acids such as oleic acid, stearic acid, isostearic acid, palmitic acid, myristic acid, behenic acid, for example, diethanolamine salts, triethanolamine salts, amino acid salts, potassium salts, sodium salts, ether carboxylic acid alkali salts, N-acylamino acid salts, N-acyl sarcosinates, higher alkyl sulfonates. Examples of cationic or amphoteric surfactants include alkyl quaternary ammonium salts, polyamines and alkyl amine salts.

Skin preparations for external use according to the present invention are preferably used in combination with amphoteric surfactants, among which imidazoline-based amphoteric surfactants are especially preferred. As used herein, the “imidazoline-based amphoteric surfactants” refer to amphoteric surfactants containing an imidazoline ring in their molecules and amphoteric surfactants having an opened imidazoline ring. Examples of imidazoline-based amphoteric surfactants include 2-alkyl-N-carboxymethyl-N-hydroxyethyl imidazolinium betaine, sodium N-cocoyl-N′-carboxylethyl-N′-hydroxyethyl ethylenediamine, disodium N-cocoyl-N′-carboxymethoxyethyl-N′-carboxymethyl ethylenediamine and disodium N-cocoyl-N′-carboxymethoxyethyl-N′-carboxymethyl ethylenediamine lauryl sulfate, among which 2-alkyl-N-carboxymethyl-N-hydroxyethyl imidazolinium betaine is especially preferred. These imidazoline-based amphoteric surfactants are commercially available under trade names such as Amphitol 20YB from Kao Corporation; ENAGICOL C-40H, CNS from Lion Corporation; LEBON 105, CIB from Sanyo Chemical Industries, Ltd.; Obazoline 662Y, 662N, 662SF, CS-65 from Toho Chemical Industry Co., Ltd; Miranol C2M-NP, Miracare 2MCA/P, Miranol ULTRAC-32 from Rhodia Nikka; Rewoteric AM2CNM, AMC from Goldschmidt AG; among which Amphitol 20YB, Obazoline 662N and Miranol ULTRAC-32 can be preferably used.

Imidazoline-based amphoteric surfactants are preferably used in the amount of 0.01-50 parts by weight, more preferably 1-30 parts by weight, even more preferably 3-5 parts by weight per part by weight of propylene glycol hyaluronate esters of the present invention. Compositions containing a propylene glycol hyaluronate ester of the present invention and an imidazoline-based amphoteric surfactant are characterized by less change in viscosity during storage because of their high stability. Especially, propylene glycol hyaluronate esters having an esterification degree of 40-60% provide higher stability as compared with lower esters.

Suitable powders for use in skin preparations for external use according to the present invention include talc, kaolin, fuller earth, gum, starch, silica, silicic acid, aluminium silicate hydrate, chemically modified aluminium magnesium silicate, sodium polyacrylate, tetraalkyl aryl ammonium smectite, trialkyl aryl ammonium smectite, ethylene glycol monostearate, sodium carboxymethylcellulose, carboxyvinyl polymers, chalk, gummy matters, ethylene glycol monostearate, ethylene glycol distearate, etc.

Suitable polyols for use in skin preparations for external use according to the present invention include glycerin, polyglycerins (such as diglycerin, triglycerin, tetraglycerin), ethylene glycol, propylene glycol, 1,3-butylene glycol, 1,4-butylene glycol, dipropylene glycol, polyethylene glycol, sorbitol, erythritol, maltotriose, threitol, sucrose, glucose, maltose, multitose, fructose, xylitose.

Other materials suitable for use in skin preparations for external use according to the present invention include vitamins such as vitamin A, vitamin B₁, vitamin B₂, vitamin B₆, vitamin B₁₂, vitamin C, vitamin D, vitamin E, vitamin K; amino acids such as proline, leucine, isoleucine, alanine, threonine, lysine, cysteine, arginine; hormones such as estrogen, pregnenolone, adrenocortical hormone; peptides such as keratin, collagen, elastin; sugars as listed above for polyols; inorganic salts such as sodium chloride, sodium hydrogencarbonate, sodium carbonate, borax, sodium sulfate, sodium sulfide, sodium thiosulfate, sodium sesquicarbonate, magnesium oxide, calcium carbonate, magnesium carbonate, potassium chloride, potassium sulfide; Streptococcus themophilus cultures; sterols such as cholesterol, provitamin D₃, campesterol, stigmastanol, stigmasterol, 5-dihydrochole-sterol, α-spinastenol, cholesterol fatty acid esters; sphingosines such as sphingosine, dihydrosphingosine, phytosphingosine, dehydrosphingosine, dehydrophytosphingosine, sphingadienine; ceramides; pseudoceramides; saponins; chitin derivatives; oligosaccharides such as maltose, xylobiose, isomaltose, lactose, sucrose, raffinose, maltotriose, xylotriose, maltotetraose, xylotetraose, maltopentaose, xylopentaose, maltohexaose, xylohexaose, maltoheptaose, xyloheptaose; acid mucopolysaccharides such as hyaluronic acid, chondroitin sulfate, dermatan sulfate, heparin, heparan sulfate; yeast extracts, etc.

Skin preparations for external use according to the present invention may further contain thickeners such as carboxyvinyl polymers, carboxymethylcellulose, polyvinyl alcohol, xanthan gum, carrageenan, alginates, propylene glycol alginate esters, gelatin; electrolytes such as sodium chloride; whitening agents such as arbutin, allantoin,vitamin E derivatives, glycyrrhizin, magnesium ascorbyl phosphate, kojic acid, pantothenic acid derivatives, placenta extract, coix seed extract, green tee, kudzu root, mulberry root, glycyrrhiza, scutellaria root, aloe, orange peel, chamomile, Ganoderma lucidum; skin protective agents such as retinol, retinol esters, retinoic acid; skin softeners such as stearyl alcohol, glyceryl monoricinoleate, mink oil, cetyl alcohol, stearic acid, coconut oil, castor oil, isostearic acid; emollients such as stearyl alcohol, glycerin monoricinoleate, glycerin monostearate, cetyl alcohol; skin penetration enhancers such as 2-methylpropane-2-ol, 2-propanol, ethyl 2-hydroxypropionate, 2,5-hexanediol, acetone, tetrahydrofuran; bioactive plant extracts such as aloe, arnica, glycyrrhiza, sage and swertia herb extracts; preservatives such as p-hydroxybenzoate esters, sodium benzoate, urea, methylparaben, ethylparaben, propylparaben, butylparaben; antiinflammatory agents such as salicylic acid; antibiotics such as triclosan; antioxidants such as α-tocopherol, butylhydroxytoluene; buffers such as triethanolamine or a combination of sodium hydroxide and lactic acid; keratolytic agents such as lactic acid, glycolic acid, malic acid, tartaric acid, citric acid; scrubbing agents such as polyethylene powder; pigments such as calcium, barium or aluminium lake, iron oxide, titanium dioxide, mica, etc.

Skin preparations for external use according to the present invention may also contain other materials depending on the purpose of use. The amount of each ingredient to be added and the method for adding it can be determined by those skilled in the art.

The following examples and test examples further illustrate the present invention. The materials, reagents, proportions, procedures or the like shown in the following examples can be appropriately changed without departing from the spirit of the present invention. Therefore, the scope of the present invention is not limited to the embodiments shown below.

### EXAMPLES

### Example 1

Preparation of Propylene Glycol Hyaluronate Esters

Sodium hyaluronate (300 g; hyaluronate FCH-200 available from Kibun Food Chemifa Co., Ltd.) was mixed with a mixed solution of 29.7 L of ethanol and 60 mL of hydrochloric acid and the mixture was stirred at room temperature for 30 minutes and then washed with ethanol/water (9:1) to give a mixture of hyaluronic acid and sodium hyaluronate.

Then, 100 g (dry weight) of this mixture was mixed with 104 mL of a solution of 6.2 equivalents of propylene oxide in ethanol/water (8:2) for esterification at 50° C. for 4 hours, followed by hyaluronate conversion as described above and further esterification at 50° C. for 8 hours. After reaction, the reaction product was washed with ethanol/water (9:1), neutralized with a solution of 1.5 equivalents of sodium acetate in ethanol/water (9:1), washed again with ethanol/water (9:1) and dried under reduced pressure at 40° C. for 2 hours to give a propylene glycol hyaluronate ester having an esterification degree of 47.6% and a limiting viscosity of 7.3 dL/g.

A plurality of propylene glycol hyaluronate esters having varying esterification degrees and limiting viscosities were prepared by the procedure described above.

### Test Example 1

Stability Test at Low pH Range Each sample was dissolved in a citrate buffer (0.2% aqueous paraben) at a sample concentration of 0.2% and stored at 50° C. for 14 days, after which viscosity change was determined at pH 3, pH 4 and pH 5. The table below shows the number of days before viscosity retention declined below 30%. Sodium hyaluronate used in the following test examples was FCH-120 available from Kibun Food Chemifa Co., Ltd. The abbreviations ED8.6 to ED90.8 refer to propylene glycol hyaluronate esters with the indices indicating esterification degrees. Viscosity was measured at 20° C. on a Brookfield rotational viscometer (Tokyo Keiki).

TABLE 1

| | pH 3 | pH 4 | pH 5 |
|---|---|---|---|
| FCH-120 | 1 | 1 | 4 |
| ED8.6 | 1 | 4 | 8 |
| ED11.0 | 4 | 4 | 8 |
| ED26.5 | >14 | >14 | >14 |
| ED34.4 | >14 | >14 | >14 |
| ED51.2 | >14 | >14 | >14 |
| ED90.8 | >14 | >14 | >14 |

The results in the table above demonstrate that propylene glycol hyaluronate esters having an esterification degree of more than about 20% exhibit high stability in low-pH systems.

### Test Example 2

Stability Test in the Presence of Cations

Each sample was added to an aqueous solution of NaCl, CaCl₂ or MgCl₂ (1.0 mol/L) at a sample concentration of 0.5% and the viscosity retention was determined after 30 minutes. The table below shows the results of evaluation in which “X” means viscosity retention of less than 20%, “Δ” means 20-30%, “◯” means 30-40% and “⊚” means more than 40%. FCH—SU refers to a low molecular weight sodium hyaluronate available from Kibun Food Chemifa Co., Ltd.

TABLE 2

| | NaCl | CaCl₂ | MgCl₂ |
|---|---|---|---|
| FCH-120 | Δ | X | X |
| FCH-SU | ◯ | ◯ | ◯ |
| ED8.6 | ⊚ | ◯ | ◯ |
| ED11.0 | Δ | Δ | Δ |
| ED34.4 | ◯ | ◯ | ◯ |
| ED51.2 | ⊚ | ⊚ | ⊚ |
| ED75.4 | ⊚ | ⊚ | ⊚ |

The results in the table above demonstrate that propylene glycol hyaluronate esters having an esterification degree of more than about 20% exhibit high stability in the presence of cations. Especially, propylene glycol hyaluronate esters having an esterification degree of more than 40% were shown to have high stability in the presence of cations.

### Test Example 3

Stability Test in the Presence of Amphoteric Surfactants

Aqueous solutions of each of Amphitol 20YB available from Kao Corporation, Miranol ULTRAC-32 available from Nikko Chemicals Co., Ltd. and Obazoline 662N available from Toho Chemical Industry Co. were prepared at 3 different concentrations of 1.0%, 1.5% and 2.5%. Each sample was added to each aqueous amphoteric surfactant solution at a sample concentration of 0.5% and adjusted to pH 3 with hydrochloric acid. The viscosity retention was determined after storage at 50° C. for 30 days. The table below shows the results of evaluation based on the number of days before viscosity retention declined below 20% in which “X” means within one day, “Δ” means within 4 days, “◯” means within 30 days and “⊚” means over 30 days. Samples used in this test example had a limiting viscosity of 18.0-19.0 dL/g.

TABLE 3

| | 20YB | | | ULTRAC-32 | | | 662N | | |
|---|---|---|---|---|---|---|---|---|---|
| | 1.0 | 1.5 | 2.5 | 1.0 | 1.5 | 2.5 | 1.0 | 1.5 | 2.5 |
| FCH-80 | X | X | X | X | X | Δ | X | X | X |
| ED18.9 | ◯ | ◯ | ⊚ | Δ | Δ | ◯ | Δ | X | X |
| ED42.2 | ⊚ | ⊚ | ⊚ | ⊚ | ⊚ | ⊚ | ◯ | ◯ | ⊚ |
| ED59.4 | ⊚ | ⊚ | ⊚ | ⊚ | ⊚ | ⊚ | ⊚ | ⊚ | ⊚ |

The results in the table above demonstrate that compositions containing a propylene glycol hyaluronate ester of the present invention and an imidazoline-based amphoteric surfactant exhibit high stability. Especially, propylene glycol hyaluronate esters having an esterification degree of more than 40% were shown to have high stability in the presence of imidazoline-based amphoteric surfactants.

### Test Example 4

Emulsifiability Test

An aqueous solution of each sample was added to a 1:1 mixed solution of distilled water and squalane at a sample concentration of 0.5% or 1.0%. Then, the mixed solution was heated to 70° C. and stirred with a hand homogenizer at 10000 rpm for 2 minutes, and allowed to cool down to room temperature to observe separation state after 4 hours. Emulsification stability was evaluated from the extent of separation according to 4 ratings of ⊚, ◯, Δ, X.

TABLE 4

Sample concentration

| | 0.5% | 1.0% |
|---|---|---|
| FCH-120 | X | X |
| ED8.6 | X | X |
| ED26.5 | ◯ | ⊚ |
| ED27.2 | ⊚ | ⊚ |
| ED34.4 | ⊚ | ⊚ |
| ED54.4 | ⊚ | ⊚ |
| ED61.0 | ◯ | ⊚ |
| ED75.4 | ◯ | ⊚ |
| ED81.7 | ◯ | ◯ |

The results in the table above demonstrate that propylene glycol hyaluronate esters having an esterification degree of more than about 20% exhibit high emulsification stability.

### Test Example 5

Emulsifiability Test at Low pH Range

An aqueous solution of each sample at a sample concentration of 0.1% was prepared and adjusted to pH 3-6 with hydrochloric acid and sodium hydroxide. To 10 g of this aqueous solution was added 10 g of squalane, and the mixed solution was heated at 70° C. for 5 minutes, then homogenized at 5000 rpm for 2 minutes and then allowed to stand at room temperature and evaluated for emulsification stability.

A series of 6 propylene glycol hyaluronate esters having esterification degrees of 13.5-59.4% and limitingviscosities of 16.55-19.0 dL/g according to the present invention were tested in comparison with a control FCH-80 to show that all of the samples according to the present invention have significantly higher emulsification stability than the control sample.

Another series of 3 propylene glycol hyaluronate esters having esterification degrees of 59.4-61.1% and limiting viscosities of 7.88-18.1 dL/g according to the present invention were tested in comparison with 3 control samples FCH—SU, 60 and 80 to show that all of the samples according to the present invention have significantly higher emulsification stability than the control samples.

Thus, propylene glycol hyaluronate esters of the present invention were shown to have high emulsification stability at pH range of 3-6.

### Test Example 6

Hydration Test

A filter paper was immersed 1 cm from one end in 50 mL each of aqueous solutions of each sample having concentrations of 0.05%, 0.1%, 0.2% and 0.5%, and allowed to stand as such for 30 minutes and then removed from the aqueous solution to measure the distance of water moved from the water level. The distance becomes longer as hydration power increases. The hydration power of each sample was evaluated according to 4 ratings ⊚, ◯, Δ, X.

TABLE 5

| | Hydration power |
|---|---|
| FCH-SY | Δ |
| ED11.0 | Δ |
| ED34.4 | ◯ |
| ED51.2 | ⊚ |
| ED81.7 | ⊚ |

The results in the table above demonstrate that propylene glycol hyaluronate esters having an esterification degree of more than about 20% exhibit high hydration power.

### Test Example 7

Hydration Test at Low pH Range

An aqueous solution of each sample at a sample concentration of 0.5% or 1.0% was prepared and adjusted to pH 3-6 with hydrochloric acid and sodium hydroxide. A vial having a diameter of 3 cm was charged with this aqueous solution and covered with a filter paper, and then placed in an incubator at 30° C. Change of the weight of the aqueous solution in each vial was measured over time and the amount of moisture evaporated off was determined to evaluate hydration.

Two propylene glycol hyaluronate esters having esterification degrees of 13.5-59.4% and limiting viscosities of 16.9-18.1 dL/g according to the present invention were tested in comparison with a control FCH-80 to show that both samples according to the present invention have significantly higher hydration power than the control sample.

Thus, propylene glycol hyaluronate esters of the present invention were shown to have high hydration power at pH range of 3-6.

### Example 2

Preparation of Beauty Lotions

Various ingredients described in the table below were mixed at room temperature and thoroughly stirred to prepare beauty lotions. In the following examples, the “active ingredient” refers to a propylene glycol hyaluronate ester having an esterification degree of 10-90% and a limiting viscosity of 3-35 dL/g.

TABLE 6

| Ingredient | Part by weight |
|---|---|
| Active ingredient | 1.0 |
| Methylparaben | 0.1 |
| Polyoxyethylene hydrogenated castor oil | 1.2 |
| Polyoxyethylene sorbitan oleate | 0.4 |
| Ethanol | 5.3 |
| Purified water | 92.0 |

### Example 3

Preparation of Powder Foundations

Various ingredients described in the table below were mixed at room temperature and thoroughly stirred to prepare powder foundations.

TABLE 7

| Ingredient | Part by weight |
|---|---|
| Active ingredient | 1.0 |
| Mica | 37.8 |
| Talc | 20.0 |
| Titanium dioxide | 12.0 |
| Kaolin | 5.0 |
| Iron oxide | 3.5 |
| Nylon powder | 8.0 |
| Octyldodecyl myristate | 2.0 |
| Neopentylglycol diisooctanoate | 2.0 |
| Sorbitan monooleate | 0.5 |
| Zinc stearate | 1.0 |
| Red oxide | 1.0 |
| Squalane | 6.0 |
| Preservative | 0.1 |
| Antioxidant | 0.1 |

### Example 4

Preparation of Whitening Powders

Various ingredients described in the table below were mixed and pulverized at room temperature to prepare whitening powders.

TABLE 8

| Ingredient | Part by weight |
|---|---|
| Active ingredient | 20.0 |
| Sucrose | 50.0 |
| Polyethylene glycol | 10.0 |
| Silica | 4.5 |
| Vitamin C | 5.0 |
| Vitamin C dipalmitate | 10.0 |
| Dye | 0.5 |

### Example 5

Preparation of Emollient Creams

After 1,3-butylene glycol and purified water described in the table below were mixed and heated to 70° C., a mixture of the remaining ingredients molten by heating was added and the emulsified particles were homogenized and cooled to prepare emollient creams.

TABLE 9

| Ingredient | Part by weight |
|---|---|
| Active ingredient | 5.0 |
| Stearyl alcohol | 6.0 |
| Stearic acid | 2.0 |
| Hydrogenated lanolin | 4.0 |
| Squalane | 9.0 |
| Octyl dodecanol | 10.0 |
| POE (25) cetyl alcohol ether | 3.0 |
| Glycerin monostearate | 2.0 |
| 1,3-Butylene glycol | 10.0 |
| Dye | 0.5 |
| Preservative | 0.1 |
| Antioxidant | 0.1 |
| Purified water | 48.3 |

### Example 6

Preparation of Cleansing Foams

Stearic acid, palmitic acid, myristic acid, lauric acid, coconut oil and preservative described in the table below were molten by heating and kept at 70° C. and a mixture of potassium hydroxide and purified water was added with stirring. Then, the remaining ingredients were added and the mixture was thoroughly stirred and then deaerated and cooled to prepare cleansing foams.

TABLE 10

| Ingredient | Part by weight |
|---|---|
| Active ingredient | 4.5 |
| Stearic acid | 10.0 |
| Palmitic acid | 10.0 |
| Myristic acid | 12.0 |
| Lauric acid | 4.0 |
| Coconut oil | 2.0 |
| Potassium hydroxide | 6.0 |
| Glycerol monostearate ester | 2.0 |
| POE (25) sorbitan monostearate | 2.0 |
| Dye | 0.5 |
| Preservative | 0.1 |
| Chelating agent | 0.2 |
| Purified water | 46.7 |

### Example 7

Preparation of Packs

Titanium oxide and talc described in the table below were thoroughly dispersed in purified water and then combined with sorbitol. The mixture was molten by heating to 70° C. and combined with the remaining ingredients and the mixture was thoroughly stirred and then deaerated and cooled to prepare packs.

TABLE 11

| Ingredient | Part by weight |
|---|---|
| Active ingredient | 4.5 |
| Polyvinyl acetate emulsion | 15.0 |
| Polyvinyl alcohol | 10.0 |
| Jojoba oil | 2.0 |
| Squalane | 2.0 |
| POE sorbitan monostearate ester | 1.0 |
| Titanium oxide | 5.0 |
| Talc | 10.0 |
| Sorbitol | 10.0 |
| Ethanol | 8.0 |
| Dye | 0.5 |
| Preservative | 0.2 |
| Purified water | 31.8 |

### Example 8

Preparation of Lipsticks

Various ingredients described in the table below were heated to 70° C. and then mixed. The mixture was thoroughly stirred and cast and then rapidly cooled to prepare lipsticks.

TABLE 12

| Ingredient | Part by weight |
|---|---|
| Active ingredient | 2.0 |
| Castor oil | 25.0 |
| Cetyl 2-ethylhexanoate | 20.0 |
| Lanolin | 10.0 |
| Isopropyl myristate ester | 10.0 |
| Candelilla wax | 9.0 |
| Solid paraffin | 8.0 |
| Carnauba wax | 5.0 |
| Beeswax | 5.0 |
| Titanium dioxide | 5.0 |
| Dye | 1.0 |

### Example 9

Preparation of Lip Creams

Active ingredient, stearic acid, stearyl alcohol and butyl stearate described in the table below were heated to 70° C. and then mixed and combined with a mixture of the remaining ingredients. The mixture was thoroughly stirred to prepare lip creams.

TABLE 13

| Ingredient | Part by weight |
|---|---|
| Active ingredient | 4.0 |
| Stearic acid | 14.0 |
| Stearyl alcohol | 8.0 |
| Butyl stearate | 10.0 |
| Propylene glycol | 10.0 |
| Glycerin monostearate | 4.0 |
| Potassium hydroxide | 1.0 |
| Antioxidant | 0.2 |
| Purified water | 48.8 |

### Example 10

Preparation of Cheek Colors

Various ingredients except for perfume and liquid paraffin described in the table below were mixed at room temperature and then sprayed with perfume and liquid paraffin and pulverized. The mixture was compression molded to prepare cheek colors.

TABLE 14

| Ingredient | Part by weight |
|---|---|
| Active ingredient | 1.5 |
| Talc | 77.8 |
| Kaolin | 9.0 |
| Zinc myristate | 5.0 |
| Pigment | 3.0 |
| Liquid paraffin | 3.0 |
| Perfume | 0.5 |
| Preservative | 0.2 |

### Example 11

Preparation of Eyeliners

Carbon black described in the table below was pulverized and then dispersed in purified water, and the remaining ingredients were mixed at room temperature to prepare eyeliners.

TABLE 15

| Ingredient | Part by weight |
|---|---|
| Active ingredient | 10.0 |
| Carbon black | 5.0 |
| Polyoxyethylene dodecyl ether | 2.0 |
| Dye | 0.5 |
| Preservative | 0.2 |
| Purified water | 82.3 |

### Example 12

Preparation of Mascaras

Iron oxide, purified water and polyacrylate ester emulsion described in the table below were mixed at 70° C. and combined with a mixture of the remaining ingredients molten by heating to 70° C. The mixture was dispersed by emulsification to prepare mascaras.

TABLE 16

| Ingredient | Part by weight |
|---|---|
| Active ingredient | 4.5 |
| Iron oxide | 10.0 |
| Polyacrylate ester emulsion | 27.0 |
| Solid paraffin | 8.0 |
| Lanolin wax | 8.0 |
| Light isoparaffin | 28.0 |
| Sorbitan sesquioleate | 4.0 |
| Dye | 0.5 |
| Antioxidant | 0.1 |
| Preservative | 0.1 |
| Purified water | 9.8 |

### Example 13

Preparation of Eyebrow Colors

Various ingredients except for powdery ingredients described in the table below were molten and mixed, and then combined with powdery ingredients. The mixture was kneaded and molded to prepare eyebrow colors.

TABLE 17

| Ingredient | Part by weight |
|---|---|
| Active ingredient | 1.0 |
| Iron oxide | 19.0 |
| Titanium oxide | 5.0 |
| Talc | 10.0 |
| Kaolin | 15.0 |
| Japan wax | 20.0 |
| Stearic acid | 10.0 |
| Beeswax | 5.0 |
| Hydrogenated castor oil | 5.0 |
| Vaseline | 4.0 |
| Lanolin | 3.0 |
| Liquid paraffin | 2.8 |
| Antioxidant | 0.1 |
| Preservative | 0.1 |

### Example 14

Preparation of Hand Creams

Various ingredients described in the table below were mixed under heating at 70° C. and thoroughly stirred to prepare hand creams.

TABLE 18

| Ingredient | Part by weight |
|---|---|
| Active ingredient | 3.0 |
| Glycerin | 20.0 |
| Urea | 2.0 |
| Monoglyceride stearate | 2.5 |
| Vaseline | 6.0 |
| Liquid paraffin | 10.0 |
| Purified water | 56.5 |

### Example 15

### Preparation of Hair Shampoos

Various ingredients described in the table below were mixed under heating at 70° C. and thoroughly stirred to prepare hair shampoos.

TABLE 19

| Ingredient | Part by weight |
|---|---|
| Active ingredient | 5.0 |
| Glycerin | 1.0 |
| Sodium polyoxyethylene lauryl sulfate | 10.0 |
| ester | 6.0 |
| Sodium lauryl sulfate ester | 3.0 |
| Coconut fatty acid diethanolamide | 0.1 |
| Sequestrant | 0.5 |
| pH modulator | 0.2 |
| Preservative | 74.2 |
| Purified water | |

### Example 16

Preparation of Hair Rinses

Various ingredients described in the table below were mixed under heating at 70° C. and thoroughly stirred to prepare hair rinses.

TABLE 20

| Ingredient | Part by weight |
|---|---|
| Active ingredient | 3.0 |
| Silicone oil | 2.8 |
| Liquid paraffin | 1.2 |
| Glycerin | 2.5 |
| Cetyl alcohol | 1.3 |
| Stearyl alcohol | 1.1 |
| Stearyltrimethylammonium | 0.6 |
| chloride | 1.0 |
| Dye | 0.2 |
| Preservative | 86.3 |
| Purified water | |

### Example 17

Preparation of Hair Lotions

Various ingredients described in the table below were mixed at room temperature to prepare hair lotions.

TABLE 21

| Ingredient | Part by weight |
|---|---|
| Active ingredient | 1.0 |
| Polyoxypropylene butyl ether | 20.0 |
| Polyoxyethylene hydrogenated castor oil | 1.0 |
| Ethanol | 50.0 |
| Perfume | 0.5 |
| Purified water | 27.5 |

### Example 18

Preparation of Bath Formulas

Various ingredients described in the table below were mixed at room temperature to prepare bath formulas.

TABLE 22

| Ingredient | Part by weight |
|---|---|
| Active ingredient | 10 |
| Sodium sulfate | 50 |
| Sodium hydrogencarbonate | 25 |
| Sodium chloride | 13 |
| Dye | 2 |

As apparent from the foregoing description, propylene glycol hyaluronate esters of the present invention are compounds showing excellent viscosity stability in low-pH systems and cation-containing systems and also showing high emulsifiability, hydration power and moisturizing effect. Therefore, skin preparations for external use according to the present invention are especially useful as moisturizers, emulsifiers for low-pH systems, emulsifiers for cation-containing systems and high-hydration emulsifiers.

### Test Example 8

Liposomes Stability Test with Propylene Glycol Hyaluronate Esters having Different Limiting Viscosity

50 mg of egg yolk lecithin (by Wako Pure Chemicals) was put into a 200-mL eggplant flask, and dissolved in 5 mL of chloroform added there. The solvent was removed through distillation under reduced pressure, and then the thin film of lipid having remained in the bottom of the eggplant flask was voltexed with 5mL of distilled water added little by little thereto to prepare a suspension of liposomes having a final concentration of 10 mg/mL. This was ultrasonicated for 30 minutes in a 21-W sonicator bath, and then a powdery sample shown in Table 23 was, as it was, added thereto to have a concentration of 10 mg/mL. Then, this was stirred as such for 2 hours at 20° C.. Phospholipase D (by Waku Pure Chemicals) was added to it to have a final concentration of 3 units, and then reacted at 37° C. for 20 minutes. Next, an EDTA solution was added to it to stop the reaction, and the amount of the released choline was measured according to a choline esterase/phenol method. Based on the measured data, the increase in the liposome stability was obtained according to the following equation, in which a indicates the released choline amount (μg) in the system with no sample added, and b indicates the released choline amount (μg) in the system with the sample added.

Liposome Stability Increase (%)=(a−b)/a×100.

Based on the liposome stability increase, the liposome stability was evaluated according to the criteria mentioned below. The result is given in Table 23.

[Criteria for Liposome Stability Evaluation]

⊚: Increase, more than 7.5%.

◯: Increase, 5.0-7.5%.

Δ: Increase, 4.0-5.0%.

X: Increase, less than 4.0%.

TABLE 23

| | Details of Substance | | | Result of |
|---|---|---|---|---|
| | | Limiting | | Liposome |
| Sample | Compound | Viscosity | Esterification | Stability |
| No. | Name | (dL/g) | Degree (%) | Evaluation |
| 8-1 | PGH | 12.1 | 19.4 | Δ |
| 8-2 | | 14.0 | 20.3 | ◯ |
| 8-3 | | 20.0 | 20.0 | ◯ |
| 8-4 | | 30.0 | 19.5 | ⊚ |
| 8-5 | | 33.2 | 18.6 | ⊚ |
| 8-6 | Sodium | 32.8 | 0.0 | Δ |
| | Hyaluronate | | | |

PGH: Propylene glycol hyaluronate ester

As in Table 23, the liposome suspensions with propylene glycol hyaluronate ester having a limiting viscosity of 14.0-33.2 dL/g added were highly stabilized. This supports the excellent liposomes-stabilizing effect of the propylene glycol hyaluronate ester of the invention, suggesting that the stabilized liposomes are widely usable in various fields for cosmetics, medicines, medicated preparations for external use, etc.

### Test Example 9

Liposomes Stability Test with Propylene Glycol Hyaluronate Esters having Different Esterification Degree

In the same manner as in Test Example 8, liposomes with 15 propylene glycol hyaluronate ester having a different esterification degree added were tested for stability. The result is given in Table 24.

TABLE 24

| | Details of Substance | | | Result of |
|---|---|---|---|---|
| | | Limiting | | Liposome |
| Sample | Compound | Viscosity | Esterification | Stability |
| No. | Name | (dL/g) | Degree (%) | Evaluation |
| 9-1 | PGH | 18.2 | 4.8 | Δ |
| 9-2 | | 17.9 | 9.4 | Δ |
| 9-3 | | 18.3 | 27.7 | ◯ |
| 9-4 | | 17.7 | 57.6 | ⊚ |
| 9-5 | | 18.0 | 61.7 | ⊚ |
| 9-6 | Sodium | 17.5 | 0.0 | Δ |
| | Hyaluronate | | | |

PGH: Propylene glycol hyaluronate ester

As in Table 24, the liposome suspensions with propylene glycol hyaluronate ester having an esterification degree of 27.7-61.7% added were highly stabilized. This supports the excellent liposomes-stabilizing effect of the propylene glycol hyaluronate ester of the invention, suggesting that the stabilized liposomes are widely usable in various fields for cosmetics, medicines, medicated preparations for external use, etc.

### Test Example 10

Emulsion Stability Test with Propylene Glycol Hyaluronate Esters having Different Limiting Viscosity

0.1 parts by weight of propylene glycol hyaluronate ester was dissolved in 49.9 parts by weight of ion-exchanged water, and the pH of the resulting aqueous solution was controlled to fall between 3 and 7 with 0.1 mol/L HCl or 0.1 mol/L NaOH added thereto. To the pH-controlled, aqueous propylene glycol hyaluronate ester solution, dropwise added was 50.0 parts by weight of an oily agent little by little while emulsified with an emulsifying machine to prepare an emulsion composition. Six types of propylene glycol hyaluronate esters each having a different limiting viscosity were combined with four different types of oily agents to prepare 24 different types of emulsion compositions in total. Thus prepared, all the emulsion compositions were stored at 50° C., and their conditions were observed. Based on the following criteria, the emulsion stability of each composition was evaluated.

[Criteria for Emulsion Stability Evaluation]

◯: The emulsion phase did not separate at all.

Δ: The emulsion phase separated but a little.

X: The emulsion phase obviously separated.

TABLE 25

| | | Details of PGH | | | | | | |
|---|---|---|---|---|---|---|---|---|
| | | | Esteri- | | | | | |
| Sam- | | Limiting | fication | | | | | |
| ple | Oily | Viscosity | Degree | Details of PGH | | | | |
| No. | Agent | (dL/g) | (%) | pH3 | pH4 | pH5 | pH6 | pH7 |
| 10-1 | Squa- | 5.2 | 18.7 | X | Δ | Δ | Δ | Δ |
| 10-2 | lene | 11.4 | 15.8 | X | ◯ | Δ | Δ | ◯ |
| 10-3 | | 13.8 | 18.1 | X | ◯ | Δ | ◯ | Δ |
| 10-4 | | 19.8 | 18.5 | ◯ | ◯ | ◯ | ◯ | ◯ |
| 10-5 | | 22.0 | 18.4 | ◯ | ◯ | ◯ | ◯ | ◯ |
| 10-6 | | 31.6 | 20.2 | ◯ | ◯ | ◯ | ◯ | ◯ |
| 10-7 | Liquid | 5.2 | 18.7 | X | Δ | Δ | Δ | X |
| 10-8 | Paraf- | 11.4 | 15.8 | X | Δ | ◯ | Δ | Δ |
| 10-9 | fin | 13.8 | 18.1 | X | Δ | ◯ | Δ | ◯ |
| 10-10 | | 19.8 | 18.5 | ◯ | ◯ | ◯ | ◯ | ◯ |
| 10-11 | | 22.0 | 18.4 | ◯ | ◯ | ◯ | ◯ | ◯ |
| 10-12 | | 31.6 | 20.2 | ◯ | ◯ | ◯ | ◯ | ◯ |
| 10-13 | Jojoba | 5.2 | 18.7 | Δ | Δ | Δ | Δ | Δ |
| 10-14 | Oil | 11.4 | 15.8 | Δ | ◯ | Δ | Δ | Δ |
| 10-15 | | 13.8 | 18.1 | Δ | ◯ | ◯ | ◯ | ◯ |
| 10-16 | | 19.8 | 18.5 | ◯ | ◯ | ◯ | ◯ | ◯ |
| 10-17 | | 22.0 | 18.4 | ◯ | ◯ | ◯ | ◯ | ◯ |
| 10-18 | | 31.6 | 20.2 | ◯ | ◯ | ◯ | ◯ | ◯ |
| 10-19 | Olive | 5.2 | 18.7 | Δ | X | X | X | X |
| 10-20 | Oil | 11.4 | 15.8 | Δ | X | Δ | Δ | Δ |
| 10-21 | | 13.8 | 18.1 | ◯ | X | ◯ | Δ | ◯ |
| 10-22 | | 19.8 | 18.5 | ◯ | ◯ | ◯ | ◯ | ◯ |
| 10-23 | | 22.0 | 18.4 | ◯ | ◯ | ◯ | ◯ | ◯ |
| 10-24 | | 31.6 | 20.2 | ◯ | ◯ | ◯ | ◯ | ◯ |

PGH: Propylene glycol hyaluronate ester

### Test Example 11

Emulsion Stability Test with Propylene Glycol Hyaluronate Esters having Different Esterification Degree

0.1 parts by weight of propylene glycol hyaluronate ester was dissolved in 49.9 parts by weight of ion-exchanged water, and the pH of the resulting aqueous solution was controlled to fall between 3 and 6 with 0.1 mol/L HCl or 0.1 mol/L NaOH added thereto. To the pH-controlled, aqueous propylene glycol hyaluronate ester solution, dropwise added was 50.0 parts by weight of squalane (by Iwase Cosfa) little by little while emulsified with an emulsifying machine to prepare an emulsion composition. Six types of propylene glycol hyaluronate esters each having a different esterification degree and one sodium hyaluronate were used to prepare 7 different types of emulsion compositions in total. Thus prepared, all the emulsion compositions were stored at 50° C., and their conditions were observed. Based on the same criteria as in Test Example 10, the emulsion stability of each composition was evaluated.

TABLE 26

| | Details of PGH | | | | | | |
|---|---|---|---|---|---|---|---|
| | | | Esteri- | | | | |
| | | Limiting | fication | Result of Emulsion | | | |
| Sample | Compound | Viscosity | Degree | Stability Evaluation | | | |
| No. | Name | (dL/g) | (%) | pH3 | pH4 | pH5 | pH6 |
| 11-1 | PGH | 16.9 | 13.5 | ◯ | ◯ | ◯ | ◯ |
| 11-2 | | 18.3 | 18.9 | ◯ | ◯ | ◯ | ◯ |
| 11-3 | | 16.6 | 35.3 | ◯ | ◯ | ◯ | ◯ |
| 11-4 | | 19.0 | 42.2 | ◯ | ◯ | ◯ | ◯ |
| 11-5 | | 16.7 | 46.5 | ◯ | ◯ | ◯ | ◯ |
| 11-6 | | 18.1 | 59.4 | ◯ | ◯ | ◯ | ◯ |
| 11-7 | Sodium | 14.2 | 0.0 | Δ | X | X | X |
| | Hyaluronate | | | | | | |

PGH: Propylene glycol hyaluronate ester

As is obvious from the result shown in Table 26, the propylene glycol hyaluronate esters exhibit an excellent emulsion-stabilizing effect. This suggests that the propylene glycol hyaluronate esters of the invention are expected to have a remarkable effect as an emulsion stabilizer and will be widely usable in various fields for cosmetics, medicines, medicated preparations for external use, etc. The stabilizing effect of these esters is obviously higher than the effect shown in the related art references mentioned hereinabove.

### Test Example 12

Skin moisturizing Test with Propylene Glycol Hyaluronate Esters having Different Limiting Viscosity

An aqueous 0.5% propylene glycol hyaluronate ester solution was prepared and applied to the inside of the forearm of four adult panelists having a healthy skin. The propylene glycol hyaluronate ester dose was 25 μg/cm² of the skin. 0.5 hours, 4 hours and 8 hours after the application, the skin conductance was measured with a device for measuring the water content of skin keratin layer (IBS' SKICON-200). In this test, tried were 6 types of propylene glycol hyaluronate esters each having a different limiting viscosity all in the form of their aqueous solutions, and distilled water with no propylene glycol hyaluronate ester therein. Based on the value of the distilled water as a standard value, the difference between the value of each aqueous ester solution and the standard value was obtained, and the conductance (corrected) of each aqueous ester solution was obtained. According to the criteria mentioned below, the skin moisturizing effect of the samples was evaluated.

[Criteria for Skin Moisturizing Effect Evaluation]

⊚: After 0.5 hours, the conductance (corrected) increased by 3.5 μS or more, and it further increased later on.

◯: After 0.5 hours, the conductance (corrected) increased by 1.5 μS or more, and it was almost on the same level later on.

Δ: After 0.5 hours, the conductance (corrected) increased by less than 1.5 μS, and it was almost on the same level later on.

X: The conductance changed little.

TABLE 27

Details of PGH

| | Limiting | | Result of Skin |
|---|---|---|---|
| Sample | Viscosity | Esterification | Moisturization |
| No. | (dL/g) | Degree (%) | Evaluation |
| 12-1 | 16.9 | 13.5 | ◯ |
| 12-2 | 18.3 | 18.9 | ◯ |
| 12-3 | 16.6 | 35.3 | ⊚ |
| 12-4 | 19.0 | 42.2 | ⊚ |
| 12-5 | 16.7 | 46.5 | ⊚ |
| 12-6 | 18.1 | 59.4 | ⊚ |

PGH: Propylene glycol hyaluronate ester

As is obvious from the result in Table 27, the aqueous solution of the propylene glycol hyaluronate ester of the invention applied to skin obviously moisturized the skin. In particular,the esters having an esterification degree of 20% or more are preferred. This suggests the applicability of the propylene glycol hyaluronate ester of the invention as a moisturizer to various fields of cosmetics, medicines, medicated preparations for external use, etc.

### Test Example 13

Test for Treating Roughened Skin

The skin of men and women panelists of 6 each was artificially roughened by applying thereto 50 μL/cm² of 5% SDS with closing the treated skin area. An emulsion shown in Table 28 was applied to the roughened skin area, twice a day. Its dose was 50 μL/cm². One day and 7 days after the start of the test, the skin conductance of the roughened site was measured with a device for measuring the water content of skin keratin layer (IBS' SKICON-200). As a result, it was confirmed that the emulsion of Sample No. 8-1 that contains propylene glycol hyaluronate ester is more effective for moisturizing and smoothing dry and rough skin than the comparative emulsions Sample No. 8-2 and Sample No. 8-3. This suggests that the propylene glycol hyaluronate ester of the invention may give excellent skin moisturizers and skin conditioners.

TABLE 28

| | Sample Number | | |
|---|---|---|---|
| Component Name | 8-1 | 8-2 | 8-3 |
| Cetanol | 0.5 | 0.5 | 0.5 |
| Vaseline | 2.0 | 2.0 | 2.0 |
| Squalene | 7.0 | 7.0 | 7.0 |
| Self-emulsifying Glycerin | 2.5 | 2.5 | 2.5 |
| Monostearate | | | |
| Polyoxyethylene (20) Sorbitan | 1.5 | 1.5 | 1.5 |
| Monostearate | | | |
| Jojoba Oil | 5.0 | 5.0 | 5.0 |
| Propylene Glycol | 5.0 | 5.0 | 5.0 |
| Glycerin | 5.0 | 5.0 | 5.0 |
| Talc | 5.0 | 5.0 | 5.0 |
| Propylene glycol hyaluronate ester | 0.1 | — | — |
| Sodium Hyaluronate | — | 0.1 | — |
| Ion-exchanged Water | 66.4 | 66.4 | 66.5 |
| Total | 100.0 | 100.0 | 100.0 |

### Test Example 14

Solubility Test

The solubility of propylene glycol hyaluronate ester was compared with that of sodium hyaluronate that is widely used in skin preparations for external use. Propylene glycol hyaluronate ester having a limiting viscosity of 28.7 dL/g and an esterification degree of 24.3%, and sodium hyaluronate having a limiting viscosity of 24.9 dL/g were prepared. 0.2 g of each of these compounds was put into a container, and 20 g of the solvent shown in Table 29 was added thereto and vigorously shaken. The resulting mixtures were kept at room temperature or 40° C. overnight, and their conditions were observed. Based on the criteria mentioned below, the solubility of the compounds was evaluated. The result is given in Table 29.

[Criteria for Solubility Evaluation]

◯: The compound completely dissolved.

Δ: The compound partly dissolved or swelled.

X: The compound did not dissolve.

TABLE 29

| | |
|---|---|
| Room Temp. | 40° C. |

| Solvent | PGH | SH | PGH | SH |
|---|---|---|---|---|
| Distilled Water | ∘ | ∘ | ∘ | ∘ |
| 60% Glycerin | ∘ | ∘ | ∘ | ∘ |
| 30% Glycerin | ∘ | ∘ | ∘ | ∘ |
| Glycerin (concentrated) | x | x | x | x |
| 30% Propylene Glycol | ∘ | ∘ | ∘ | ∘ |
| 60% Propylene Glycol | ∘z | ∘ | ∘ | ∘ |
| Propylene Glycol (concentrated) | x | x | x | x |
| 30% 1,3-Butylene Glycol | ∘ | ∘ | ∘ | ∘ |
| 60% 1,3-Butylene Glycol | ∘ | ∘ | ∘ | ∘ |
| 1,3-Butylene Glycol (concentrated) | x | x | x | x |
| 20% Ethanol | ∘ | ∘ | ∘ | ∘ |
| 80% Ethanol | x | x | x | x |
| Liquid Paraffin (Molesco White P-70) | x | x | x | x |
| Liquid Paraffin (Molesco White | x | x | x | x |
| P-350P) | | | | |
| Squalene | x | x | x | x |
| Olive Oil | x | x | x | x |
| Middle-chain Fatty Acid Triglyceride | x | x | x | x |
| (Coconade MT) | | | | |
| Middle-chain Fatty Acid Triglyceride | x | x | x | x |
| (Coconade RK) | | | | |
| Isopropyl Myristate | x | x | x | x |
| Dimethylpolysiloxane | x | x | x | x |
| Methylphenylpolysiloxane | x | x | x | x |

PGH (propylene glycol hyaluronate) has a limiting viscosity of 31.6 dL/g and an esterification degree of 20.2. SH (sodium hyaluronate) has a limiting viscosity of 30.0 dL/g.

As in Table 29, the solubility of the propylene glycol hyaluronate ester of the invention is comparable to that of sodium hyaluronate. Unsubstituted hyaluronic acid has a high degree of hydrability. However, the degree of hydrability of hyaluronic derivatives substitutedwith a hydrophobic group often lowers, and the solubility of the derivatives in various solvents may vary. However, the solubility of the propylene glycol hyaluronate ester of the invention changes little from that of hyaluronic acid. Therefore, the propylene glycol hyaluronate ester of the invention is usable as a substitute for sodium hyaluronate that is widely used in skin preparations for external use. For example, the formulation of skin preparations for external use that contains sodium hyaluronate may directly apply to skin preparations for external use that contains the propylene glycol hyaluronate ester of the invention. The invention does not require any additional investigation of designing different formulations of skin preparations for external use with the solubility of the ester being taken into consideration, and this is an advantage of the invention.

### Test Example 15

Test for Operability in Emulsification

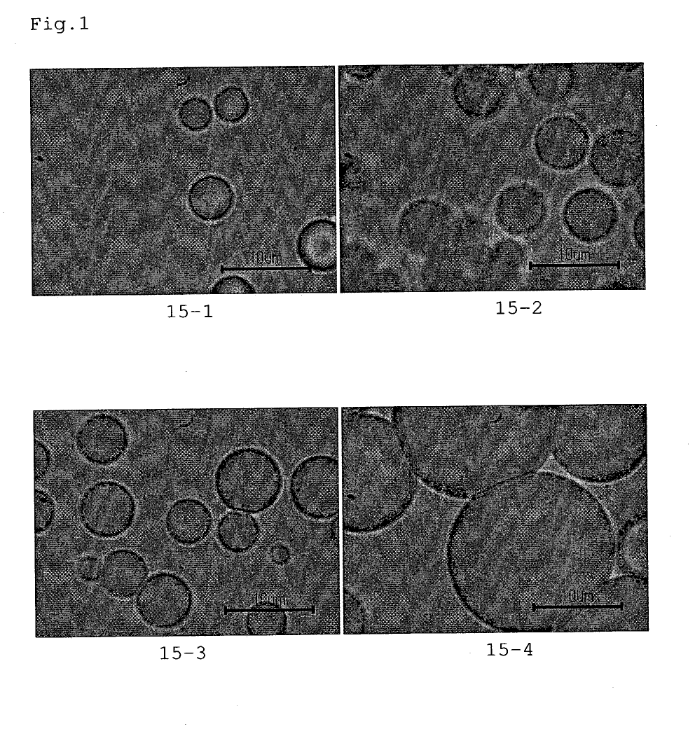

0.1 parts by weight of propylene glycol hyaluronate ester was dissolved in 49.9 parts by weight of ion-exchanged water, and 50.0 parts by weight of squalane (by Iwase Cosfa) was added thereto and emulsified with an emulsifying machine to prepare an emulsion composition. The number of revolution of the emulsifying machine for emulsifying the components was 15,000 rpm, and the emulsification time was 30 seconds. Four types of emulsion compositions were prepared, each containing propylene glycol hyaluronate ester having a different limiting viscosity (Table 30). Thus prepared, the emulsion compositions were observed with a microscope, and the microscopic photographs of the dispersed particles are shown in FIG. 1.

TABLE 30

| | |
|---|---|
| Details of PGH | Result of |

| | Limiting | | Emulsification |
|---|---|---|---|
| Sample | Viscosity | Esterification | Operability |
| No. | (dL/g) | Degree (%) | Evaluation |
| 15-1 | 17.9 | 20.3 | good |
| 15-2 | 23.2 | 22.9 | good |
| 15-3 | 30.0 | 22.6 | good |
| 15-4 | 35.4 | 15.1 | not good |

PGH: Propylene glycol hyaluronate ester

From FIG. 1, it is understood that the size of the emulsified particles that were prepared at the same revolution and within the same period of time becomes larger with the increase in the limiting viscosity of the ester used. When emulsion compositions are prepared, it is necessary that the particle size of the emulsified particles is as small as possible. In addition, the samples in which the ester has a high limiting viscosity must be stirred for a longer period of time or by the use of an emulsifying machine of higher performance in order that the emulsified particles may be thinner. This is unfavorable in view of the efficiency in emulsification operation. From the result as above, it is recognized that the limiting viscosity of the propylene glycol hyaluronate ester that enables good emulsification operation falls between 14 and 35 dL/g, preferably between 14 and 33.5 dL/g.

What is claimed is:

1. A propylene glycol hyaluronate ester having a limiting viscosity of 14-35 dL/g and an esterification degree of 10-90%.

2. A propylene glycol hyaluronate ester having a limiting viscosity of 14-33.5 dL/g and an esterification degree of 10-90%.

3. The propylene glycol hyaluronate ester of claim 1 which has an esterification degree of 10-65%.

4. The propylene glycol hyaluronate ester of claim 1 which has an esterification degree of 15-65%.

5. The propylene glycol hyaluronate ester of claim 1 which has an esterification degree of 15-40%.

6. The propylene glycol hyaluronate ester of claim 1 which has an esterification degree of 20-40%.

7. A composition comprising the propylene glycol hyaluronate ester of claim 1 and an imidazoline-based amphoteric surfactant.

8. The composition of claim 8 wherein the imidazoline-based amphoteric surfactant is selected from the group consisting of 2-alkyl-N-carboxymethyl-N-hydroxyethyl imidazolinium betaine, sodium N-cocoyl-N′-carboxylethyl-N′-hydroxyethyl ethylenediamine, disodium N-cocoyl-N′-carboxymethoxyethyl-N′-carboxymethyl ethylenediamine and disodium N-cocoyl-N′-carboxymethoxyethyl-N′-carboxymethyl ethylenediamine lauryl sulfate.

9. The composition of claim 7 wherein the imidazoline-based amphoteric surfactant is 2-alkyl-N-carboxymethyl-N-hydroxyethyl imidazolinium betaine.

10. An emulsifier comprising the propylene glycol hyaluronate ester of claim 1.

11. A composition for stabilizing liposome comprising the propylene glycol hyaluronate ester of claim 1.

12. A skin preparations for external use comprising the propylene glycol hyaluronate ester of claim 1.

13. A composition comprising liposome and the propylene glycol hyaluronate ester of claim 1.

14. A skin preparations for external use comprising liposome and the propylene glycol hyaluronate ester of claim 1.

15. A method for stabilizing liposome which comprises adding the propylene glycol hyaluronate ester of claim 1 to a composition comprising liposome.

16. A skin conditioning method which comprises applying a composition containing the propylene glycol hyaluronate ester of claim 1 to the skin.

17. A skin moisturizing method which comprises applying a composition containing the propylene glycol hyaluronate ester of claim 1 to the skin.

18. A rough-skin treatment method which comprises applying a composition containing the propylene glycol hyaluronate ester of claim 1 to the skin.
